(19) United States
(12) Patent Application Publication
(10) Pub. No.: US 2026/0364173 A1
(43) Pub. Date: Nov. 19, 2026

(54) BIOELECTRIC CONTROL OF MACROPHAGES USING EXCITED NANOSTRUCTURES

(71) Applicants: Michael Naughton, Waltham, MA (US); Krzysztof Kempa, Chestnut Hill, MA (US)

(72) Inventors: Michael Naughton, Waltham, MA (US); Krzysztof Kempa, Chestnut Hill, MA (US)

(73) Assignee: The Trustees of Boston College, Chestnut Hill, MA (US)

(21) Appl. No.: 19/133,914

(22) PCT Filed: Nov. 30, 2023
(86) PCT No.: PCT/US2023/081900

§ 371 Date: May 29, 2025

Related U.S. Application Data
(60) Provisional application No. 63/428,764, filed on Nov. 30, 2022.

(51) Int. Cl. A61K 41/00; A61K 33/00

(52) U.S. Cl. CPC A61K 41/0028; A61K 33/00

(57) ABSTRACT

Disclosed are methods and systems for inducing a change in a polarization state of one or more macrophages. The method includes introducing one or more nanostructures into a microenvironment of one or more macrophages. The method further includes inducing a change in the polarization state of the one or more macrophages by exciting the one or more nanostructures with an energy source external to the microenvironment. Microphages play a key role in the human immune response and controlling macrophage behavior, through selective control of macrophage polarization, has broad applications for treating human disease, injury, or other ailments.

### CROSS-REFERENCE TO RELATED APPLICATIONS

This application claims the benefit of the filing date of U.S. Provisional Patent Application No. 63/428,764, filed Nov. 30, 2022, which is hereby incorporated by reference in its entirety.

### FIELD OF THE DISCLOSURE

The present disclosure generally relates to controlled polarization of macrophages. More specifically, the present disclosure is directed to methods and systems for inducing a change in macrophage polarization state using nanostructures excited by an external energy source.

### BACKGROUND

A macrophage is a type of myeloid cell and is part of the innate immune system, active in the presence of inflammation and infection. Macrophages are highly plastic cells whose phenotype is influenced by both physiological processes—such as embryogenesis—and pathological conditions, such as tissue repair, cancer, infection, allergies, and chronic inflammation. Historically, macrophage polarization included two states: 1) a “classical” pro-inflammatory activation state (M1), and 2) an “alternative” or anti-inflammatory activation state (M2).

Currently, it is generally appreciated that macrophage polarization occurs along a continuous spectrum of cell functions. This polarization depends on biochemical factors such as expression of cell surface markers, secreted cytokines and chemokines, phagocytotic capability and transcriptional statuses. Importantly, macrophage polarization can be a major determinant of the outcome of infection, injury, and inflammation.

While macrophages play key protective roles against infection and cancer, certain pathogens can block their protective effects (e.g. Mycobacterium tuberculosis). Moreover, there is evidence that macrophages can be polarized to M2-like states within tumor microenvironments contributing to immunosuppressive and difficult-to-treat cancers such as pancreatic adenocarcinomas. In some other cancers (e.g. lung cancers) inappropriate recruitment and activation of proinflammatory macrophages can actually drive the development of tumors by secreting proinflammatory cytokines, like IL-6, IL-1, TNF-α. The ability to control macrophage polarization could be used to develop novel therapies aimed at preventing these corruptions of the immune response.

Current methods for controlling macrophage polarization generally focus on techniques for biochemically treating the macrophages, for example, with IFN-γ or IL-4. These biochemical treatment methods are often costly and time-consuming and/or cannot be used in vivo. Moreover, such approaches have largely failed to generate therapeutically translatable effects due to the plasticity of the macrophage cells and an inability to elicit longer term repolarization and status training.

Accordingly, there still exists a need in the art for methods and systems capable of controlling macrophage polarization in a manner that is time-efficient, effective over longer time-periods, and can be utilized in vivo.

### SUMMARY OF THE DISCLOSURE

The present disclosure is generally directed to methods and systems for inducing macrophage polarization using nanostructures excited by an external energy source. This disclosure is based, at least partially, on Applicants' realization and appreciation that nanostructures introduced into the macrophage microenvironment can be excited by an external energy source to produce a stimulus that is local to the macrophages. This local stimulus can then be selectively controlled based on the application of the external energy source to induce a change in polarization, and consequent behavior, of the macrophages.

Generally, one aspect of this disclosure relates to a method for inducing a change in a polarization state of one or more macrophages. The method includes introducing one or more nanostructures into a microenvironment of the one or more macrophages. The method further includes inducing a change in the polarization state of the one or more macrophages by exciting the one or more nanostructures with an energy source external to the microenvironment.

In some embodiments, introducing the one or more nanostructures includes injecting the one or more nanostructures into a tissue of a patient.

In various embodiments, the one or more nanostructures include nanoparticles, nanopowders, nanotubes, and/or nanowires.

In various embodiments, exciting the one or more nanostructures includes generating a voltage on the one or more nanostructures.

In some embodiments, the one or more nanostructures are made, at least partially, of piezoelectric materials. In some variants of these embodiments, the energy source includes an ultrasound source, and the one or more nanostructures are configured to generate a voltage in response to excitation by the ultrasound source. In particular embodiments, the one or more nanostructures include nanoparticles of barium titanate.

In some embodiments, the one or more nanostructures are made, at least partially, of photovoltaic materials. In some variants of these embodiments, the energy source includes a light source, and the one or more nanostructures are configured to generate a voltage in response to excitation by the light source. In particular embodiments, the one or more nanostructures include silicon p-n junction nanowires. In various embodiments, the light source is a wearable device configured to be worn around a patient's wrist. In various embodiments, the light source excites the one or more nanostructures with infrared light.

Various embodiments include monitoring the microenvironment using medical imaging.

In various embodiments, the one or more nanostructures are coated, at least partially, with one or more materials configured to promote uptake of the one or more nanostructures into the one or more macrophages.

In various embodiments, the one or more nanostructures are coated, at least partially, with an insulator.

In some embodiments, the one or more nanostructures are made, at least partially, of magnetic materials and the one or more nanostructures are configured to release one or more therapeutic compounds in response to excitation by RF signals from the energy source.

In particular embodiments, inducing a change in the polarization state includes inducing a change in the one or more macrophages from a pro-inflammatory M1 state to an anti-inflammatory M2 state or inducing a change in the one or more macrophages from an anti-inflammatory M2 state to a pro-inflammatory M1 state.

In some additional embodiments, inducing a change in the polarization state includes inducing a change in the one or more macrophages from an undifferentiated M0 state to a pro-inflammatory M1 state or inducing a change in the one or more macrophages from the undifferentiated M0 state to an anti-inflammatory M2 state.

In various embodiments, the method further includes inducing the creation of polarized macrophages.

Another aspect of the present disclosure relates to treating a patient using the method for inducing a change in macrophage polarization.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

FIG. 1 is an illustration of a macrophage microenvironment according to some aspects of the present disclosure;

FIG. 2 is a diagram of macrophage polarization according to some aspects of the present disclosure;

FIG. 3 is an illustration of nanostructures within a macrophage microenvironment being excited by an external energy source according to some aspects of the present disclosure;

FIG. 4 is an illustration of nanostructures coated with a material configured to promote cellular uptake of the nanostructures into the macrophages according to some aspects of the present disclosure;

FIG. 5A is an illustration of nanostructures being excited by an ultrasound source according to some aspects of the pending disclosure;

FIG. 5B is an illustration of macrophage polarization induced by a voltage generated on nanostructures excited by an ultrasound source according to some aspects of the present disclosure;

FIG. 6A is an illustration of nanowires being excited by a light source according to some aspects of the pending disclosure;

FIG. 6B is an illustration of macrophage polarization induced by a voltage generated on nanowires excited by a light source according to some aspects of the pending disclosure;

FIG. 7 is an illustration of a nanowire coated with an insulator according to some aspects of the pending disclosure;

FIG. 8A is an illustration a magnetic nanopowder being excited by an energy source according to some aspects of the pending disclosure;

FIG. 8B is an illustration of macrophage polarization induced by therapeutic chemicals released by a magnetic nanopowder;

FIG. 9 illustrates the introduction of nanostructures into a microenvironment being monitored with medical imaging;

FIG. 10 illustrates a method for inducing a change in polarization of one or more macrophages.

### DETAILED DESCRIPTION OF EMBODIMENTS

The present disclosure is generally directed to methods and systems for inducing a change in the polarization state of one or more macrophages. For the purposes of this disclosure, “polarization” refers to a change in shape, structure, or function of a cell, as the term is used by those of ordinary skill in fields relating to cell biology. This meaning is distinct from the concept of electrical polarization. This disclosure contemplates various techniques for inducing a change in the polarization state of one or more macrophages, including techniques that expose the macrophages to nanostructures made with piezoelectric or photovoltaic materials, or containing therapeutic compounds. Macrophages play a key role in the human immune response, and macrophage polarization can be a major determinant of the outcome of infection, injury, and inflammation. The ability to modulate the behavior of macrophages and macrophage-like cells, such as dendritic cells, with temporal and spatial control, could be utilized in treatments for a number of human diseases and ailments, including cancer, tumorigenesis, rheumatoid arthritis, obesity, acute lung injury, spinal cord injury, cystic fibrosis, insulin resistance, acute respiratory distress syndrome, tissue repair/wound healing, and many others.

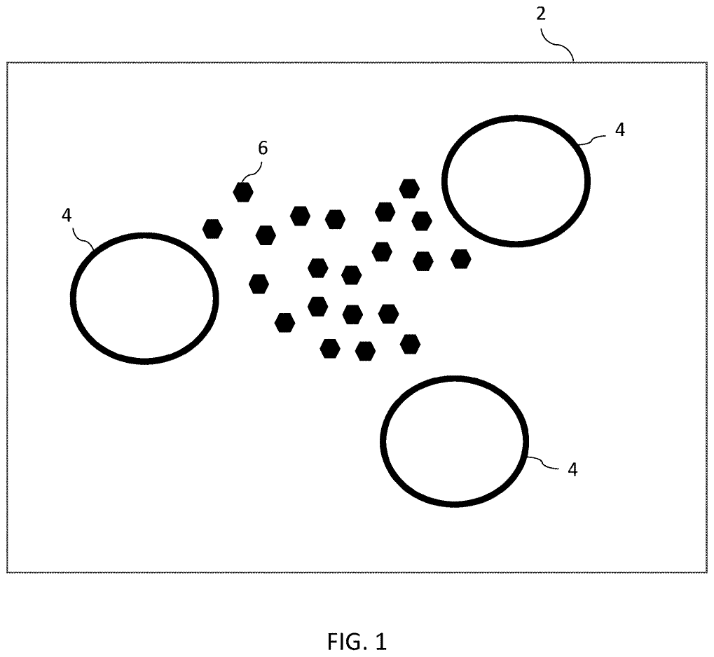

FIG. 1 is an illustration of a microenvironment 2 including one or more macrophages 4, according to some aspects of the present disclosure. Microenvironment 2 includes the local environment surrounding macrophages 4. Such a local environment can include, for example, biochemical signals surrounding macrophages 4, the extracellular matrix, or other cells. Microenvironment 2, for example, may be within the tissue of a patient.

Macrophages 4 include highly mobile white blood cells that serve several important immune system functions. For example, macrophages 4 may help to mediate the innate immune response, and, by acting as antigen presenting cells, also help active T-helper cell subsets in the adaptive branch of the immune system. Macrophages 4 are highly phagocytotic and are capable of ingesting cellular debris and foreign substances. Macrophages 4 include heterogenous and versatile cells that can undergo differentiation (phenotypical and/or functional variations) in response to changes in microenvironment 2.

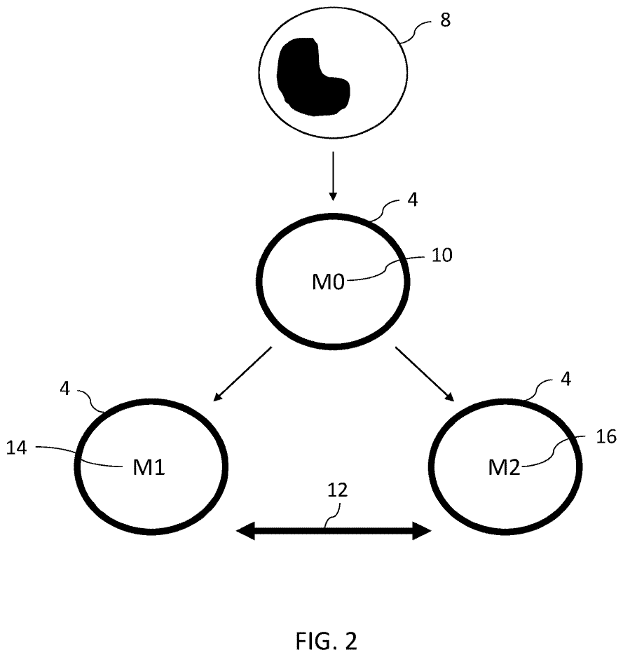

Macrophages 4 include terminally differentiated cells of the mononuclear phagocyte system which encompasses dendritic cells, circulating blood monocytes, and committed myeloid progenitor cells in bone marrow. FIG. 2 illustrates progression from a monocyte 8 to a polarized macrophage according to some aspects of the present disclosure. Monocyte 8 includes a type of white-blood capable of differentiating into a macrophage. Monocyte 8 may be produced by bone marrow and migrates to damaged or infected tissue as part of the immune response. Monocyte 8 can undergo differentiation to replenish macrophage populations resident to the affected tissue. Notably, differentiation of monocytes is not the only way of forming macrophages. Macrophages are also regularly produced as part of self-sustaining resident tissue populations.

FIG. 2 illustrates monocyte 8 differentiate into a macrophage polarized in the M0 state 10. In the M0 state 10, the macrophage is undifferentiated. In response to changes in microenvironment 2, M0 state 10 macrophage can change polarization along a spectrum 12 of states between an M1 state 14 and an M2 state 16. The macrophage polarization state includes not just M1 state 14 and M2 state 16 that form the ends of spectrum 12, but also M0 state 10 and the intermediate states along spectrum 12. Macrophages towards the M1 side of the spectrum are functionally pro-inflammatory and anti-microbial, while macrophages towards the M2 side of the spectrum are functionally anti-inflammatory. For example, a polarization state that is positioned along the M1 side of spectrum 12, but closer to the center than the M1 end would exhibit some pro-inflammatory and anti-microbial properties, but less so than a polarization state that is positioned further along the M1 side of spectrum 12. As another example, if a macrophage was changed from a state positioned slightly along the M1 side of spectrum 12 to a state positioned slightly along the M2 side of spectrum 12, then that macrophage would change from exhibiting weakly pro-inflammatory properties to weakly anti-inflammatory properties. Macrophage polarization is characterized by a complex set of factors including expression of cell markers, secreted cytokines and chemokines, and transcription and epigenetic pathways. Macrophage polarization is not fixed, as macrophages are sufficiently plastic to integrate multiple signals, such as those from microbes, damaged/diseased tissues, and those indicating healthy tissue microenvironments. Techniques to reshape macrophage polarization (i.e., converting M0 to M2, converting M1 into M2, etc.) is a promising therapeutic modality in several infectious and human inflammatory diseases.

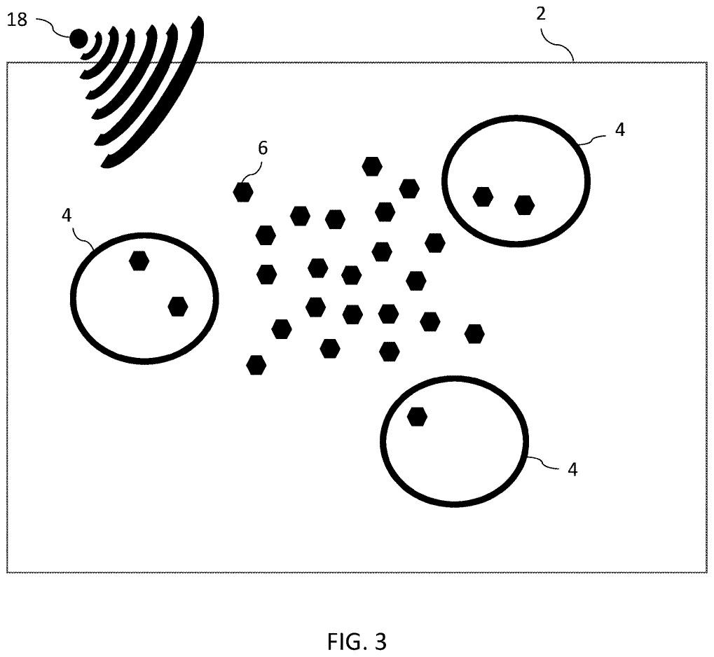

FIG. 1 further illustrates a plurality of nanostructures 6 introduced into microenvironment 2. This could involve, for example, injecting nanostructures 6 into the tissue of a patient. Nanostructures 6 can take the form of nanoparticles, nanopowders, nanotubes, nanowires, etc., wherein the spatial dimensions in at least two directions are generally smaller than about 1 micrometer. Nanostructures 6 are configured to induce a change in polarization of macrophages 4 upon excitation by an energy source that is external to microenvironment 2. FIG. 3 illustrates nanostructures 6 being excited by such an energy source 18. Energy source 18, for example, may be a device external to a patient's body, while microenvironment 2 is in tissue located within the patient's body. As exemplified further below, there are several possible techniques for exciting nanostructures 6 to induce this change of polarization.

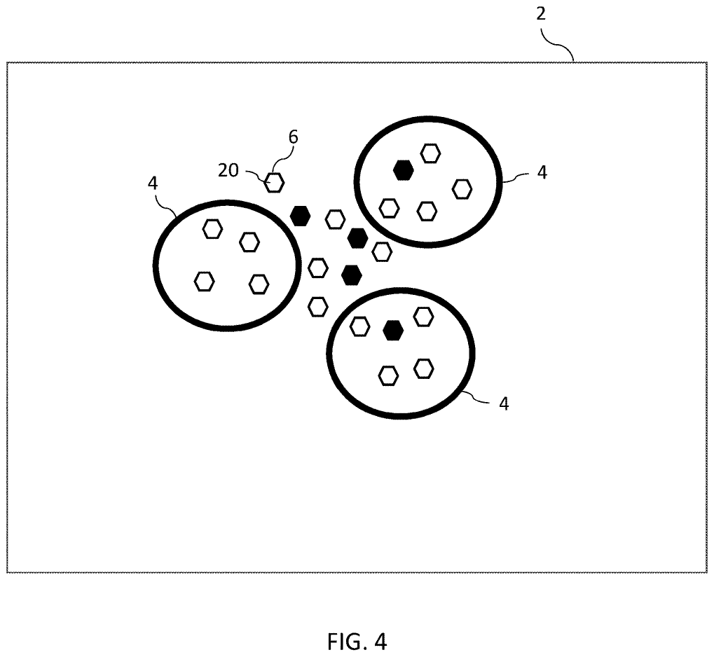

FIG. 4 illustrates nanostructures 6 coated, at least partially, with one or more materials 20 configured to promote uptake of the nanostructures 6 into macrophages 4. This could include coating nanostructures 6 with materials such as sugars or gum Arabic, or PEGylating nanostructures 6 with polyethylene glycol. Even in cases where macrophages 4 fail to uptake (i.e., consume) nanostructures 6, macrophages 4 will still gravitate towards and surround these foreign materials so that nanostructures 6 can affect a local change in microenvironment 2 once excited. Notably, as further illustrated in FIGS. 3 and 4, even in the absence of materials 20, there is still uptake of nanostructures 6 into macrophages 4. This is due to the fact that macrophages 4 regularly consume foreign matter as part of their immune function.

One way that energy source 18 can excite nanostructures 6 to induce a change in polarization of macrophages 4 is by introducing a voltage local to microenvironment 2 with nanostructures 6. This local electric potential results in a current density that acts on voltage-sensitive ion channels in the cell membranes of macrophages 4. These electrical signals serve as biophysical cues that modulate diverse aspects of cell behavior. Cell membrane voltage can influence a variety of cell behaviors, including distinguishing cell identity, dictating single cell-level nutrient transport, controlling mitotic division, and directing organism-scale morphological patterning.

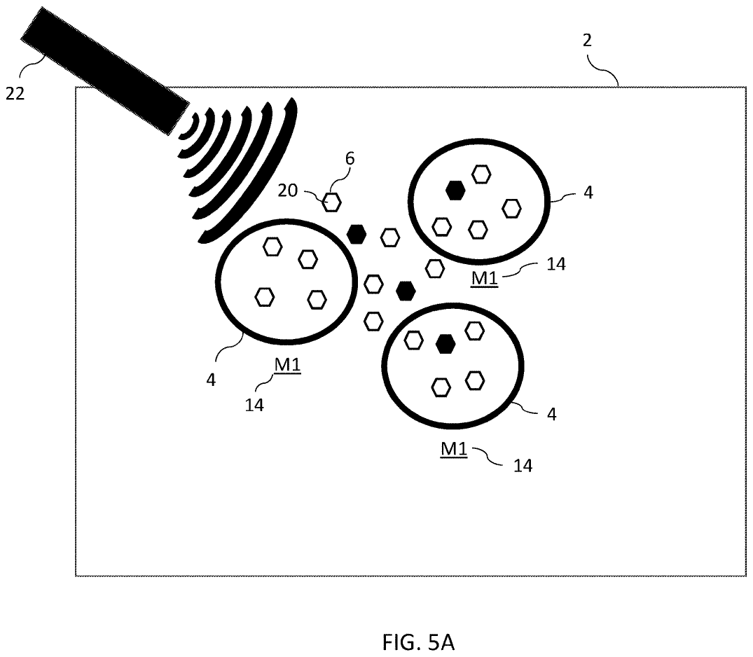

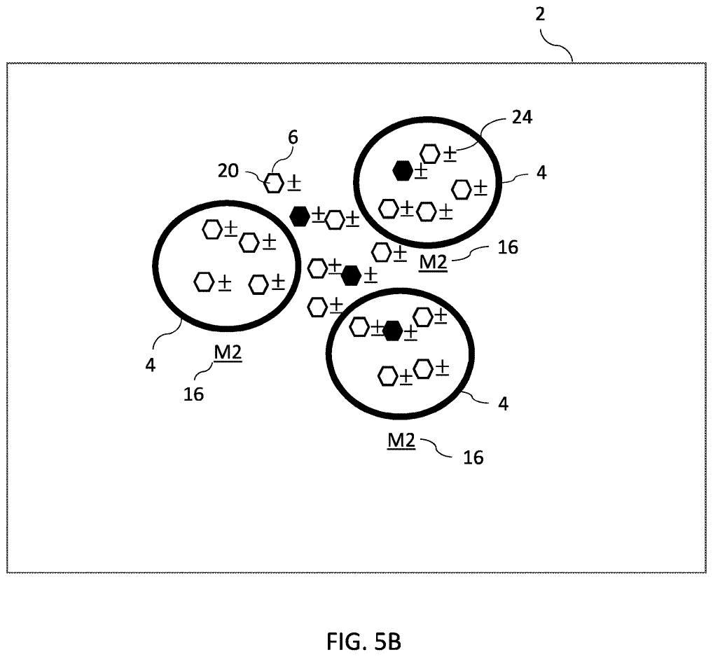

FIGS. 5A and 5B illustrate a voltage 24 being induced on nanostructures 6 using ultrasound source 22. In this example, nanostructures 6 are made of piezoelectric materials. A piezoelectric material is a material able to convert mechanical energy (via stress produced by a physical force) into electrical energy, and vice versa. The piezoelectric effect results from the linear electromechanical interaction between mechanical and electrical states, generally found in crystalline materials with no inversion symmetry. The piezoelectric materials include solid-state crystals, some ceramics, and biological matter such as bone, DNA, and proteins. Ultrasound is sound having a frequency larger than that which can be heard by humans (i.e., greater than 20 kHz). Sound is energy in the form of pressure wave moving through a carrier medium which can include air, liquids, or solids. When an ultrasound pressure wave generated from ultrasound source 22 is incident upon nanostructures 6 made from piezoelectric materials, the wave exerts mechanical stress or pressure on nanostructures 6. Due to the piezoelectric properties of nanostructures 6, mechanical stress causes electrical charges within the material to separate, thereby creating a potential difference or voltage 24 across nanostructures 6. Voltage 24 could be, for example, approximately 10⁻² V. Given that typical transmembrane potentials are on the scale of approximately 10⁻¹ V, voltage 24 represents a significant perturbation to macrophages 4. Some nonlimiting examples of piezoelectric materials that can be prepared or processed as nanoparticles are barium titanate BaTiO₃ (“BT”), lead zirconate titanate Pb[ZrₓxTi₁₋ₓ]O₃ (“PZT”), and β-phase polyvinylidene difluoride —{C₂H₂F₂}ₙ— (“β-PVDF). As prepared, BT powder is made up of clumped nanoparticles, such that dispersion is required. Some examples of appropriate dispersion protocols include dispersing the BT into polyethylene glycol PEG with sonication and centrifuge. Some examples of implementations for ultrasound source 22 include a sonoporator system or piezoelectric transducers that generate mechanical disturbances by applying electrical signals to piezoelectric materials (such as in an ultrasound probe). FIGS. 5A and 5B illustrate voltage 24 formed by excitation of nanostructures 6 inducing a change in polarization of macrophages 4 from M1 state 14 to M2 state 16.

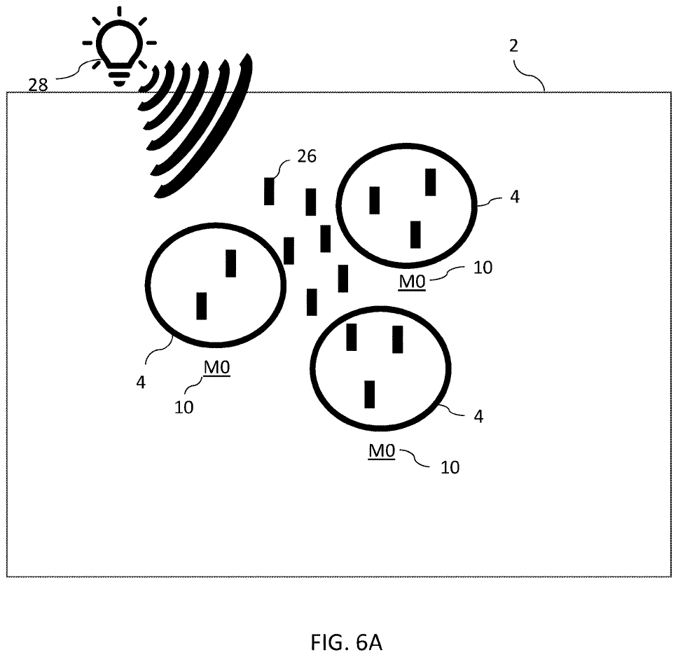

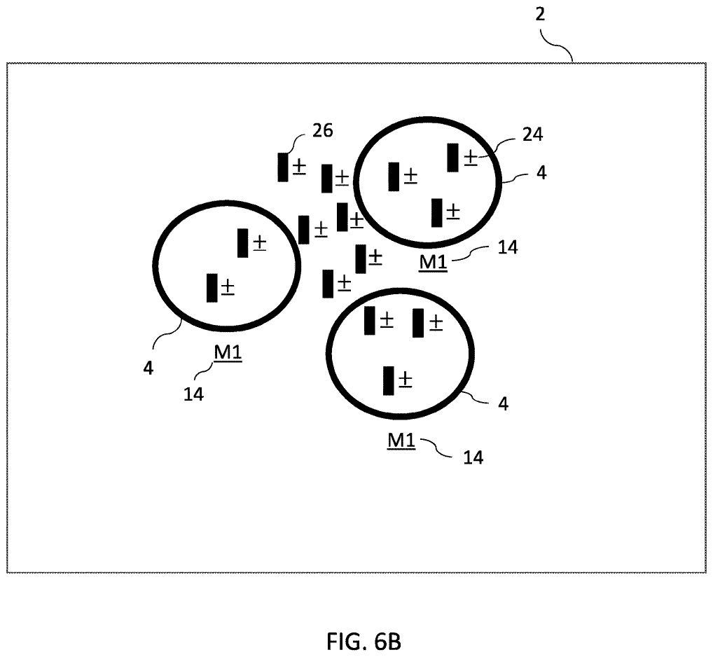

FIGS. 6A and 6B illustrate a voltage 24 being induced on nanowires 26 using a light source 28. Light source 28 is configured to excite nanowires 26 by emitting light, for example, with a frequency/wavelength in the visible to infrared range of the electromagnetic spectrum. In this example, nanowires 26 are made of photovoltaic materials. A photovoltaic material is a material able to convert light energy into electrical energy. The photovoltaic effect results from the exchange of energy between photons of light (typically in the visible and infrared ranges) and electronic states generally found in semiconductors. Specifically, this exchange is a result of the electrons within the photovoltaic material absorbing energy from photons (that make-up the visible and/or infrared light) emitted from light source 28. The absorbed energy causes the electrons to jump from states in the semiconducting material's valence band to previously unoccupied states in the semiconducting material's conduction band. Once in the conduction band, these energized electrons can migrate out of the photovoltaic material, aided by the internal electric field of a p-n junction configuration, resulting in an electric current I at a voltage V, thus providing electrical power I×V. Essentially any semiconductor with a quantum band gap in the eV range is considered photovoltaic. An example of an appropriate photovoltaic material for the nanostructures of the present disclosure is a silicon p-n junction nanowire. Light source 28, may be configured to emit light with a wavelength in the infrared range. Infrared light has the advantage of deeper penetration lengths, making them better able to reach nanostructures within the tissue of a patient. In particular, exciting the nanostructures at near infrared wavelengths (700 nm-1,300 nm) has the advantage of being able to penetrate human tissue, while also having greater excitation energy than light at far-infrared wavelengths. As shown in FIGS. 6A and 6B, energy emitted from light source 28 is used to excite photovoltaic nanowires 26. This optical excitation creates a voltage 24 on nanowires 26, due to their photovoltaic characteristics, which induces a change in the polarization of macrophages 4. As an example, FIGS. 6A and 6B show a change in polarization of macrophages 4 from undifferentiated M0 state 10 to M1 state 14.

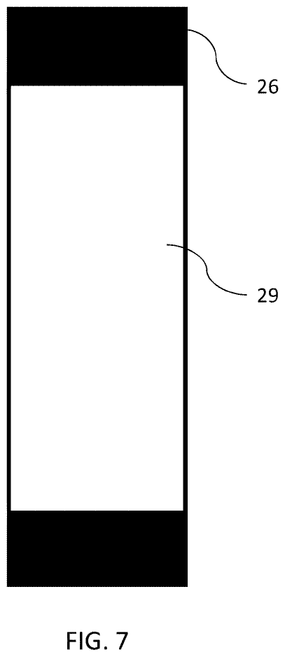

FIG. 7 illustrates nanowire 26 coated with insulator 29. FIG. 7 shows insulator 29 coating the walls, but not ends of nanowire 26. This has the advantage of better directing the electric field distribution of nanowire 26 upon excitation by light source 28. Insulator 29, for example, can be formed from atomic layer deposition of Al₂O₃ onto nanowire 26.

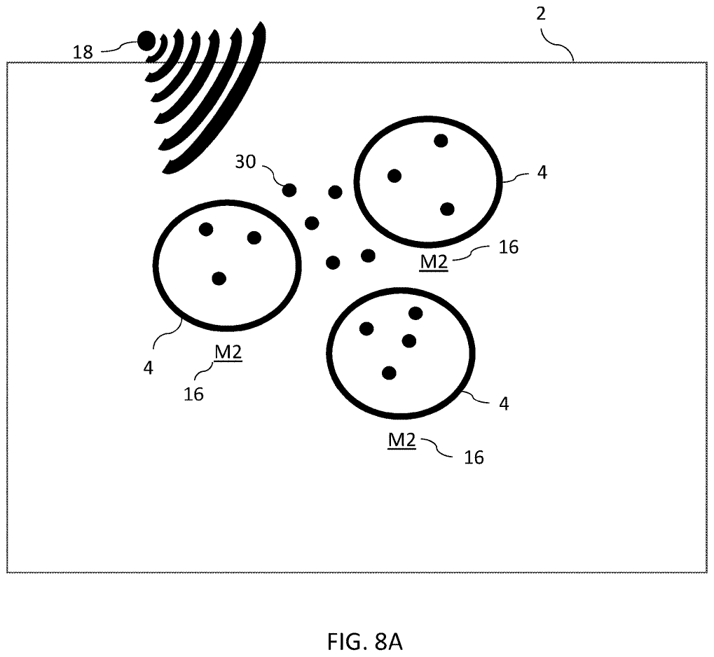

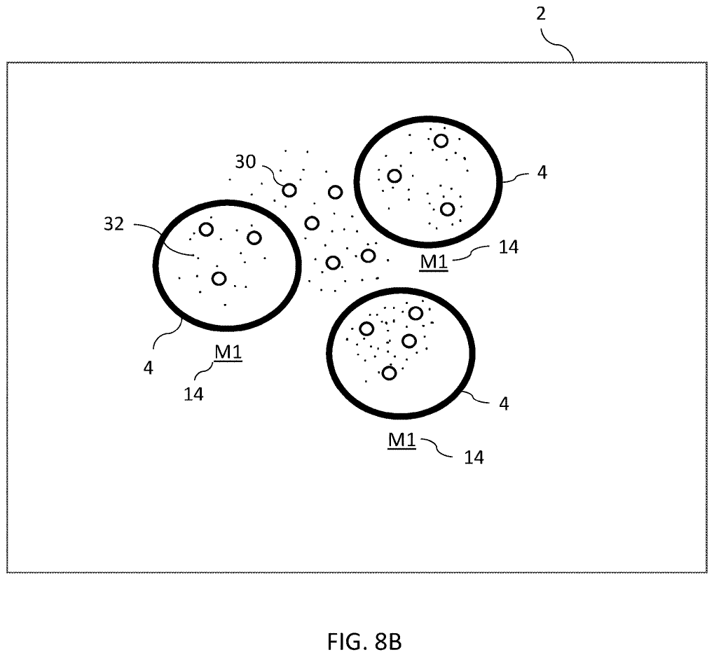

FIGS. 8A and 8B illustrate yet another way that energy source 18 can excite nanostructures 6 to induce a change in polarization of macrophages 4. FIGS. 8A and 8B show nanopowder 30 made from magnetic materials. An example of suitable magnetic materials include Iron Oxide Fe₃O₄ and Fe₂O₃ with a diameter of 5 nm-200 nm. Nanopowder 30 is conjugated with therapeutic compounds 32 configured to induce a change in polarization of macrophages 4. Examples of chemicals suitable for therapeutic compounds 32 include IFN-γ, lipopolysaccharides, IL-4, IL-3, toll-like receptor ligands, IL-1β, glucocorticoids, IL-10 and/or TGF-β. In this example, energy source 18 emits RF energy. This RF energy includes alternating magnetic fields which the magnetic materials in nanopowder 30 absorb, while the surrounding biological materials in microenvironment 2 do not, resulting in greater biological penetration. The absorption of RF energy heats up the conjugated magnetic materials and therapeutic compounds 32 that make up nanopowder 30, eventually causing nanopowder 30 to release therapeutic compounds 32 into microenvironment 2. This local release of therapeutic compounds 32 induces a change in polarization of macrophages 4. As an example, FIGS. 8A and 8B show a change in polarization of macrophages 4 from M2 state 16 to M1 state 14.

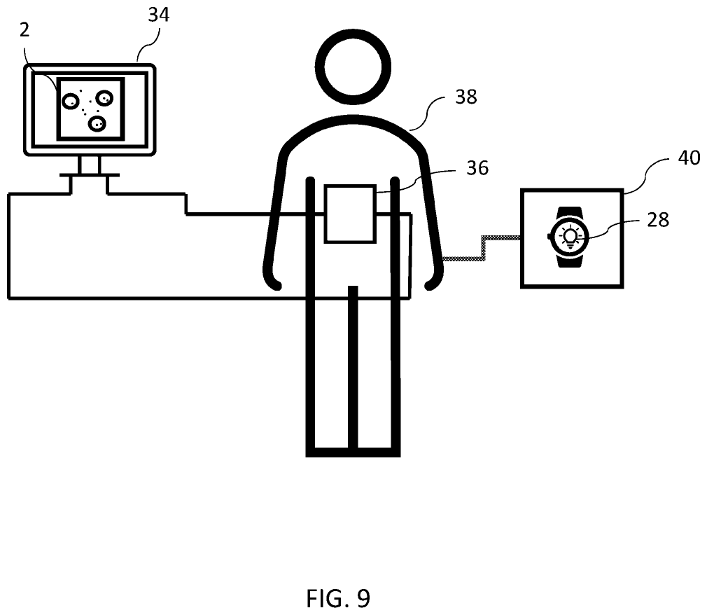

FIG. 9 illustrates the nanostructure-based polarization of macrophages 4 being monitored with medical imaging 34. Medical imaging 34 is used to monitor an area of interest 36 of patient 38. Area of interest 36 can include tissue of patient 38. Microenvironment 2 is located within area of interest 36 and continuously or periodically displayed using medical imaging 34. Medical imaging 34 may include techniques such as positron emission tomography PET or magnetic resonance imaging MRI. When PET is used for medical imaging 34, PET-active radiotracers (e.g., ¹⁸F-PEG₃) can be conjugated with nanostructures 6. PET imaging then visualizes the ¹⁸F, which is co-located with nanostructures 6. This enables directed applications of energy source 18 (e.g., via focused ultrasound when using ultrasound source 22) onto nanostructures 6 for localized activation. Such a process could similarly involve incorporating PET-active tracers into nanowires 26 made from photovoltaic materials. This would enable the location of nanowires 26 to be tracked via PET scan and then light, including visible and infrared light, can be better directed to excite nanostructures 26. In another example of using medical imaging 34 to monitor polarization of macrophages 4, piezo-electric nanostructures 6 can be coated with magnetic (superparamagnetic, ferrimagnetic, ferromagnetic) materials (or vice versa) to form hybrid magnetic-piezoelectric complexes. The magnetic component functions as an MRI contrast agent for visualization and the piezoelectric component, when activated by ultrasound, induces a change in polarization in macrophages 4.

FIG. 9 further illustrates a wearable device 40. Wearable device 40 may be implemented, for example, as a smart watch configured to be worn around the wrist of patient 38. Wearable device 40 includes light source 28 used to excite nanostructures 6 made from photovoltaic materials. In the example shown in FIG. 8, wearable device 40 is configured to emit visible light to provide periodic or continuous shallow therapy to patient 38.

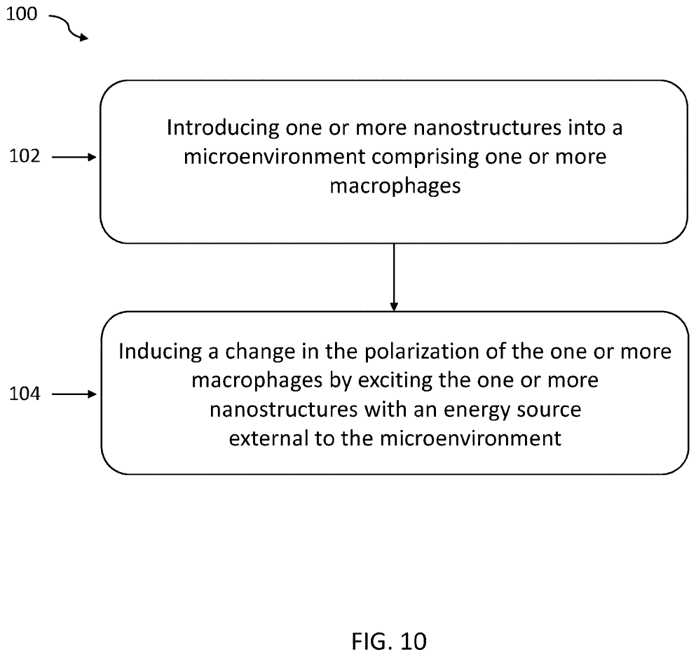

FIG. 10 illustrates a method 100 for inducing changes in macrophage polarization. Step 102 of method 100 includes introducing one or more nanostructures into a microenvironment comprising one or more macrophages. This step can be performed by introducing previously discussed nanostructures 6 into microenvironment 2 containing macrophages 4. This can involve, for example, injecting nanostructures 6 into the tissue of patient. Step 104 of method 100 includes inducing a change in the polarization of the one or more macrophages by exciting the one or more nanostructures with an energy source external to the microenvironment. This step can be performed by exciting the previously discussed nanostructures 6 with energy source 18 in order to induce a change in polarization of macrophages 4.

Macrophages 4 play an important role in the human immune response, and in particular, in controlling inflammation. Selective macrophage polarization can be used for a variety of patient treatments for example, by promoting or reducing inflammation as needed or promoting the creation of newly polarized macrophages.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The above-described examples of the described subject matter can be implemented in any of numerous ways. For example, some aspects can be implemented using hardware, software, or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

In various implementations, a processor or controller can be associated with one or more storage media (generically referred to herein as “memory,” e.g., volatile and non-volatile computer memory such as ROM, RAM, PROM, EPROM, and EEPROM, floppy disks, compact disks, optical disks, magnetic tape, Flash, OTP-ROM, SSD, HDD, etc.). In some implementations, the storage media can be encoded with one or more programs that, when executed on one or more processors and/or controllers, perform at least some of the functions discussed herein. Various storage media can be fixed within a processor or controller or can be transportable, such that the one or more programs stored thereon can be loaded into a processor or controller so as to implement various aspects as discussed herein. The terms “program” or “computer program” are used herein in a generic sense to refer to any type of computer code (e.g., software, firmware, or microcode) that can be employed to program one or more processors or controllers.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some examples, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While various examples have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the examples described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings is/are used. Those skilled in the art will recognize or be able to ascertain using no more than routine experimentation, many equivalents to the specific examples described herein. It is, therefore, to be understood that the foregoing examples are presented by way of example only and that, within the scope of the appended claims and equivalents thereto, examples can be practiced otherwise than as specifically described and claimed. Examples of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

Although various embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

What is claimed is:

1. A method (100) for inducing a change in a polarization state of one or more macrophages (4), the method comprising:
introducing (102) one or more nanostructures (6) into a microenvironment (2) comprising the one or more macrophages (4); and
inducing (104) a change in the polarization state of the one or more macrophages (4) by exciting the one or more nanostructures (6) with an energy source (18) external to the microenvironment (2).

2. The method (100) of claim 1, wherein introducing the one or more nanostructures (6) comprises injecting the one or more nanostructures (6) into a tissue of a patient (36).

3. The method (100) of claim 1, wherein the one or more nanostructures (6) comprise nanoparticles, nanopowders, nanotubes, and/or nanowires.

4. The method (100) of claim 1, wherein exciting the one or more nanostructures (6) comprises generating a voltage (24) on the one or more nanostructures (6).

5. The method (100) of claim 1, wherein the one or more nanostructures (6) are made, at least partially, of piezoelectric materials.

6. The method (100) of claim 5, wherein the energy source (18) comprises an ultrasound source (22), and the one or more nanostructures (6) are configured to generate a voltage (24) in response to excitation by the ultrasound source (22).

7. The method (100) of claim 5, wherein the one or more nanostructures (6) comprise nanoparticles of barium titanate.

8. The method (100) of claim 1, wherein the one or more nanostructures (6) are made, at least partially, of photovoltaic materials.

9. The method (100) of claim 8, wherein the energy source (18) comprises a light source (28), and the one or more nanostructures (6) are configured to generate a voltage (24) in response to excitation by the light source (28).

10. The method (100) of claim 8, wherein the one or more nanostructures (6) comprise silicon p-n junction nanowires (26).

11. The method (100) of claim 8, wherein the light source (24) is a wearable device (40) configured to be worn around a patient's wrist.

12. The method (100) of claim 8, wherein the light source (28) excites the one or more nanostructures (6) with visible or infrared light.

13. The method (100) of claim 1, further comprising monitoring the microenvironment (2) using medical imaging (34).

14. The method (100) of claim 1, wherein the one or more nanostructures (6) are coated, at least partially, with one or more materials (20) configured to promote uptake of the one or more nanostructures (6) into the one or more macrophages (4).

15. The method (100) of claim 1, wherein the one or more nanostructures (6) are coated, at least partially, with an insulator (29).

16. The method (100) of claim 1, wherein the one or more nanostructures (6) are made, at least partially, of magnetic materials and the one or more nanostructures (6) are configured to release one or more therapeutic compounds (32) in response to excitation by RF signals from the energy source (18).

17. The method (100) of claim 1, wherein inducing a change in the polarization state comprises inducing a change in the one or more macrophages (4) from a from a pro-inflammatory M1 state (14) to an anti-inflammatory M2 state (16) or inducing a change in the one or more macrophages (4) from an anti-inflammatory M2 state (16) to a pro-inflammatory M1 state (14).

18. The method (100) of claim 1, wherein inducing a change in the polarization state comprises inducing a change in the one or more macrophages (4) from an undifferentiated M0 state (10) to a pro-inflammatory M1 state (14) or inducing a change in the one or more macrophages (4) from the undifferentiated M0 state (10) to an anti-inflammatory M2 state (16).

19. The method (100) of claim 1, further comprising inducing the creation of polarized macrophages (4).

20. Treating a patient (38) using the method (100) according to claim 1.
